(19) United States
(12) Patent Application Publication
(10) Pub. No.: US 2003/0138935 A1
(43) Pub. Date: Jul. 24, 2003

(54) Selective deactivation of yeast in corn wet milling process slurries

(76) Inventor: William J. Palardy, Doylestown, PA (US)

Correspondence Address: WEGMAN, HESSLER & VANDERBURG, 6055 ROCKSIDE WOODS BOULEVARD, SUITE 200, CLEVELAND, OH, 44131, US

(21) Appl. No.: 10/055,182

(22) Filed: Jan. 23, 2002

(51) Int. Cl. C12N 1/20

(52) U.S. Cl. 435/252.9; 435/253.6

(57) ABSTRACT

A method of treating an aqueous medium of the type having yeast and lactic acid producing bacteria therein, such as a corn wet milling process, is disclosed. An effective amount of octanoic acid is added to the medium to reduce the yeast content in the aqueous medium while leaving the lactic acid producing bacteria viable.

### FIELD OF THE INVENTION

The invention relates to treatment of corn wet milling process slurries to reduce yeast content therein. One aspect of the invention relates to selective inhibition of deleterious microflora in the slurry while beneficial microflora, such as lactic acid producing bacteria, are allowed to remain viable.

### BACKGROUND OF THE INVENTION

The primary objective of corn wet milling is the separation of the four main constituents of the kernel, namely starch, gluten (primarily corn protein), germ, and fiber (primarily hull). The first major process step is preparation for mechanical separation by soaking the kernels for 24-50 hours in warm (approximately 125° F.) water containing SO₂ at a pH of about 3-5, preferably about 4. Not all of the water used in the steeping process is absorbed by the corn. The excess water contains soluble and insoluble materials liberated during steeping, as well as microbes produced during and essential to the steeping process. These steepwater solids are typically concentrated in evaporators, producing “heavy steepwater,” which is subsequently added to corn fiber, dried, and sold as animal feed.

Microflora produced in the steeping process can include both desirable (e.g., lactobacillus) and undesirable (e.g., yeast) organisms. The lactic acid produced by these desirable microorganisms is generally recognized as crucial in assuring optimal separation in downstream wet milling processes. They also enhance the nutritional content and value of the slurry or evaporated steepwater that may be later used as an animal feed source.

The undesirable microorganisms compete with the desirables for food, etc., and degrade the efficiency of the steeping process. Since many of the microorganisms (desirable and undesirable) persist in the steepwater even after concentration in an evaporator, and since the yeast also produces gas (CO₂), heavy steepwater is prone to foaming even in unagitated conditions (e.g., when stored in railcars). This latter phenomenon can result in sanitary problems from overflow of storage tanks and railcars, as well as result in the production of a rubbery residue in the vessels.

It is therefore an object to provide an effective treatment that, when admitted to a corn wet milling slurry, can reduce or inhibit growth of undesirable microorganisms, such as yeast and mold. It is an even more desirable goal to provide an effective treatment that selectively reduces or inhibits growth of these undesirable microorganisms while, at the same time, not harming desirable lactic acid producing bacteria, such as lactobacillus.

### SUMMARY OF THE INVENTION

One aspect of the invention pertains to a method for reducing yeast and other gas producing microorganisms present in a corn wet milling slurry by adding an effective amount for the purpose of octanoic acid to the slurry. Preferably, the slurry comprises corn wet milling steepwater having a pH of about 3-5.

The octanoic acid treatment may be added in an amount of about 1-500 ppm of octanoic acid based upon one million parts per volume of the slurry. More preferably, from about 10 to about 100 ppm of the treatment is added with about 10 ppm to about 50 ppm being presently even more preferred. The octanoic acid may be added separately or in combination with other treatment chemicals such as a biosurfactant.

Another aspect of the invention pertains to the selective inhibition of harmful yeast organisms in the slurry while lactic acid producing bacteria, such as lactobacillus are left, substantially unabated, in the process slurry.

Other objects and advantages of the invention will be apparent from the following description and the appended claims.

### DETAILED DESCRIPTION

The present inventor discovered that octanoic acid, added to corn wet milling steepwater, effectively reduced yeast and gas producing microorganism content of the steepwater slurry. Additionally, it was found that while the treatment was effective in reducing the gas producing microbes, it did not reduce the content of the beneficial lactic acid-producing bacteria in the test medium. This latter fact is important as it suggests that the corn wet mill slurry can be treated in accordance with the invention to reduce the undesirable yeast and gas producing microbe content without adversely affecting the beneficial lactic acid producing bacteria.

From about 1-500 ppm of the treatment can be added to the slurry based upon the volume of the slurry. Preferably, the treatment is added in an amount of about 10-100 ppm, with 10-50 ppm appearing, at present, to be even more preferred.

In addition to its use in conjunction with corn wet milling steepwater as set forth above and in the examples, any corn wet milling process slurry may benefit from the invention. Additionally, the treatment could, perhaps, be utilized in other industrial applications where selective deactivation of yeast (and other similar microbes, e.g., fungi) may be desirable. One example of such an application is in a bacterial pharmaceutical fermentation broth.

Octanoic acid (a.k.a. caprylic acid) is commercially available from a plurality of sources including Spectrum Chemical Corp. and Witco Chemicals. As stated in U.S. Pat. No. 5,234,719, it may be obtained by saponification and subsequent distillation of coconut oil. It is a colorless, oily liquid having a mp of 16° C. and a bp of 237.9° C. It is slightly soluble in water and is soluble in alcohol and ether.

The invention will be described further herein in the following examples which are useful for illustrative purposes and should not be construed as a limitation or provide any reason to narrow the scope of the claimed invention.

### EXAMPLES

Procedure

All tests were conducted using heavy steepwater samples provided by a major U.S. corn wet milling facility.

The focus of experimentation was to:

Verify and quantify the reduction in gas production (i.e., yeast activity) when this material was added to heavy steepwater samples; and

Identify and validate the selectivity of octanoic acid in killing yeast but not other desirable organisms at the intended dosages of this material.

A Flask Pressure Profile method was developed and used to measure changes in gas production. The sample of heavy steepwater was added to an Erlenmeyer flask, then placed into a water bath at 100° F. and agitated slowly (30-40 rpm). The flask was connected to a U-tube manometer filled with distilled water. After zeroing, manometer readings were taken every 30 minutes for 6 hours. An increase in flask pressure indicated gas-producing microbial activity. Tests using octanoic acid were performed in duplicate at 0 ppm (control), 10 ppm, 25 ppm, and 50 ppm (active).

To confirm the concept that octanoic acid is selective in killing only yeast but not desirable bacteria, microbiological activity was assessed by examining separate culture plates prepared using two different agars. Plates prepared using Potato-Dextrose Agar (PDA) were used to assess the fate of yeast in the present of octanoic acid, as PDA is known to be specific to the propagation of yeast and mold. Plates prepared using Sabourad-Dextrose Agar (SDA) were used to determine the fate of “desired” bacteria as SDA is specific for both yeast and lactic acid producing bacteria. Several serial dilutions (10¹ to 10⁷) of heavy steepwater samples were prepared for the pour plates at 0 ppm (control), 10 ppm, 25 ppm, and 50 ppm (active) of octanoic acid, then agitated in a 100° F. shaking water bath for 4 hours. Plates were similarly prepared using the same samples after agitation for 24 hours. All plates were incubated at 30° F. for 4 days before performing measurements. In addition to inspecting the plates for microbial activity, aliquots of each sample (after 4 hours and after 24 hours) were examined under a microscope for visual speciation.

Results

It has been discovered that octanoic acid effectively reduces all gas-producing microorganisms in corn wet milling heavy steepwater at all of the applied dosages. This is demonstrated in the results of the Flask Pressure Profile study, depicted in Table 1 below:

TABLE 1
Flask Pressure Profile Results for Heavy Steepwater Treated with Octanoic Acid

| Time, | Manometer readings, in H₂O, at time = t, hours | | | | | | | | | | | | | |
|---|---|---|---|---|---|---|---|---|---|---|---|---|---|---|
| hours | 0 | 0.5 | 1 | 1.5 | 2 | 2.5 | 3 | 3.5 | 4 | 4.5 | 5 | 5.5 | 6 | 6.5 |
| Control | 0 | 2.5 | 2.5 | 2.5 | 2.5 | 2.5 | 2.5 | 2.0 | 1.5 | 1.0 | * | * | * | * |
| Control | 0 | 1.75 | 1.75 | 1.75 | 1.25 | 1.0 | 0.75 | 0.50 | 0.25 | 0.0 | 0.0 | 0.5 | 0.75 | 1.0 |
| 10 ppm | 0 | 0.25 | 0.0 | 0.0 | 0.0 | 0.0 | 0.0 | 0.0 | 0.0 | 0.0 | * | * | * | * |
| Octanoic | | | | | | | | | | | | | | |
| Acid | | | | | | | | | | | | | | |
| 10 ppm | 0.0 | 0.0 | 0.0 | 0.0 | 0.0 | 0.0 | 0.0 | 0.0 | 0.0 | 0.0 | 0.0 | 0.0 | 0.0 | 0.0 |
| Octanoic | | | | | | | | | | | | | | |
| Acid | | | | | | | | | | | | | | |
| 25 ppm | 0.0 | 0.25 | 0.0 | 0.0 | 0.0 | 0.0 | 0.0 | 0.0 | 0.0 | 0.0 | 0.0 | 0.0 | 0.0 | 0.0 |
| Octanoic | | | | | | | | | | | | | | |
| Acid | | | | | | | | | | | | | | |
| 50 ppm | 0.0 | 0.0 | 0.0 | 0.0 | 0.0 | 0.0 | 0.0 | 0.0 | 0.0 | 0.0 | 0.0 | 0.0 | 0.0 | 0.0 |
| Octanoic | | | | | | | | | | | | | | |
| Acid | | | | | | | | | | | | | | |

* = measurement not taken

There is a clear increase in vessel pressure in those samples without octanoic acid. This increase in pressure is the direct result of gas produced by microbiological activity, as all manometers were zeroed and sealed to the flasks at the beginning of the experiments. The decrease in sealed flask pressure starting at approximately 2.5 hours is apparently the dissolution of the CO₂ (produced by the microorganisms) into the water contained in the manometer.

The first cultural plate studies were used to determine the existence and extent of microbiological activity in the heavy steepwater itself. The results for both types of agar produced significant levels of microbiological activity, and confirm that both yeast and lactic acid producing bacteria are present. These experiments validated both the feasibility of the method and assured field process samples, without synthetic doping with pure cultures, would produce dependable results.

It was discovered that octanoic acid, in the test concentrations from 10-50 ppm, selectively kills the yeast in heavy steepwater yet the treatments do not measurably decrease the activity of lactobacillus. This is evidenced in the pour-plate culture studies for both 4 hour and 24 hour incubations, following the procedures described above. The results in Table 2 show that PDA plates for treated samples exhibit virtually no microbial activity, while both the PDA plates for untreated samples and SDA plates for all samples (untreated and treated) indicate constant or increasing microbiological activity levels.

TABLE 2
Culture Plate Results for Heavy Steepwater Treated with Octanoic Acid

| Sample | | | | | |
|---|---|---|---|---|---|
| Incubation | | | Microscopic | Plate Showing | Plate Showing |
| Time, hrs. | Treatment | ppm | Evaluation | activity, PDA | activity, SDA |
| 4 | Control | 0 | Bacilli and yeast | 10² | 10² |
| 4 | Octanoic Acid | 10 | Bacilli | — | 10² |
| 4 | Octanoic Acid | 25 | Bacilli | — | 10¹ |
| 4 | Octanoic Acid | 50 | Bacilli | — | 10² |
| 24 | Control | 0 | Bacilli and yeast | 10² | 10² |
| 24 | Octanoic Acid | 10 | Bacilli | — | 10² |
| 24 | Octanoic Acid | 25 | Bacilli | — | 10² |
| 24 | Octanoic Acid | 50 | Bacilli | — | 10² |

As used herein, the phrase “corn wet milling slurry” shall refer to slurries, dispersions, suspensions, mixtures, and/or solutions containing any corn constituent and water. The phrase “aqueous medium” shall be broadly construed to include any water based slurry, dispersion, suspension, mixture, and/or solution.

While the methods herein described include a preferred embodiment and alternative embodiments of this invention, it is to be understood that the invention is not limited to specific details of the disclosed embodiments, and that changes may be made therein without departing from the scope of the invention which is defined in the appended claims.

What is claimed is:

1. Method of reducing yeast and mold present in a corn wet milling slurry comprising adding to said slurry an effective amount for the purpose of octanoic acid.

2. Method as recited in claim 1 wherein said slurry comprises corn wet milling steepwater.

3. Method as recited in claim 1 wherein said step of adding comprises adding from about 1 ppm to 500 ppm of said octanoic acid to said slurry.

4. Method as recited in claim 3 wherein said step of adding comprises adding from about 10 ppm to 100 ppm of said octanoic acid to said slurry.

5. Method as recited in claim 3 wherein said step of adding comprises adding from about 10 ppm to about 50 ppm of said octanoic acid to said slurry.

6. Method as recited in claim 2 wherein said steepwater has a pH of about 3 to 5.

7. Method of treating a corn wet milling slurry of the type having yeast and lactic acid producing bacterium therein, said method comprising adding octanoic acid to said slurry, said octanoic acid being effective to reduce the amount of said yeast in said slurry while leaving, substantially unabated, said lactic acid producing bacterium in said slurry.

8. Method as recited in claim 7 wherein said lactic acid producing bacterium is lactobacillus.

9. Method as recited in claim 8 wherein said corn wet milling slurry comprises acidic corn wet milling steepwater having an acidic pH of between about 3 to 5.

10. Method as recited in claim 9 comprising adding about 1 ppm to 500 ppm of said octanoic acid to said steepwater.

11. Method as recited in claim 10 comprising adding about 10 ppm to 100 ppm of said octanoic acid to said steepwater.

12. Method as recited in claim 11 comprising adding about 10 ppm to 50 ppm of said octanoic acid to said steepwater.

13. Method of treating an aqueous medium of the type having yeast and lactic acid producing bacteria therein, said method comprising adding to said aqueous medium an effective amount of octanoic acid to reduce the yeast content in said medium while leaving said lactic acid producing bacteria viable.

14. Method as recited in claim 13 wherein about 10 to 100 ppm of octanoic acid is added to said medium.

15. Method as recited in claim 14 wherein said aqueous medium comprises a pharmaceutical fermentation broth.
